(19) United States
(12) Patent Application Publication
(10) Pub. No.: US 2024/0336918 A1
(43) Pub. Date: Oct. 10, 2024

(54) METHODS OF CONTROLLING RNA BINDING PROTEIN ACTIVITY

(71) Applicant: The Regents of the University of California, Oakland, CA (US)

(72) Inventors: Eugene Yeo, La Jolla, CA (US); Frederick Tan, Encinitas, CA (US)

(21) Appl. No.: 18/577,881

(22) PCT Filed: Jul. 8, 2022
(86) PCT No.: PCT/US2022/036459

§ 371 Date: Jan. 9, 2024

Related U.S. Application Data
(60) Provisional application No. 63/219,916, filed on Jul. 9, 2021.

(51) Int. Cl. C12N 15/113

(52) U.S. Cl. CPC C12N 15/113; C12N 2310/14; C12N 2310/531

(57) ABSTRACT

Provided are methods of inhibiting activity of a RNA binding protein in a cell that include (a) generating a RNA binding N protein (RBP) binding agent comprising an RNA molecule comprising a binding site for the RNA binding protein; and (b) delivering the RBP binding agent into the cell, wherein the RBP binding agent binds to the RNA binding protein, thereby inhibiting the activity of the RNA binding protein in the cell.

### CROSS-REFERENCE TO RELATED APPLICATIONS

This application claims priority to U.S. Provisional Patent Application No. 63/219,916, filed on Jul. 9, 2021, the content of which is incorporated by reference in its entirety.

### BACKGROUND

The activity of RNA binding proteins (RBPs) is often reduced by gene expression knockdown (e.g., using siRNAs or antisense oligonucleotides). However, controlling the activity of RBPs and identifying the binding properties of an RBP in a transcriptome-wide manner remains technically challenging.

### SUMMARY

Provided herein are methods of inhibiting activity of a RNA binding protein (RBP) in a cell, the method comprising: (a) generating a RNA binding protein (RBP) binding agent comprising an RNA molecule comprising a binding site for the RNA binding protein; and (b) delivering the RBP binding agent into the cell, wherein the RBP binding agent binds to the RNA binding protein, thereby inhibiting the activity of the RNA binding protein in the cell.

In some embodiments, the RNA molecule is a single strand RNA.

In some embodiments, the RBP binding agent comprises one or more binding sites for the RNA binding protein. In some embodiments, the RNA binding protein binds to the one or more binding sites with low affinity. In some embodiments, the RNA binding protein binds to the one or more binding sites with high affinity. In some embodiments, the RBP binding agent comprises a single binding site for a single RNA binding protein. In some embodiments, the RBP binding agent comprises a plurality of binding sites for a plurality of the same RNA binding protein. In some embodiments, the RBP binding agent comprises a plurality of binding sites for a plurality of different RNA binding proteins.

In some embodiments, the RBP binding agent further comprises a pseudoknot at each of the 5′ and 3′ ends. In some embodiments, the RBP binding agent further comprises a SCNMV Exo element and a MALAT1 Pseudoknot, respectively on the 5′ and 3′ ends.

In some embodiments, the RBP binding agent comprises a synthetic gene. In some embodiments, the RBP binding agent is expressed by using a DNA expression vector. In some embodiments, the DNA expression vector comprises a promoter. In some embodiments, the promoter is a TRE promoter.

In some embodiments, the delivering comprises transfection, electroporation, or a virus-based delivery. In some embodiments, the delivering comprises a virus-based delivery. In some embodiments, the virus-based delivery comprises an adeno-associated virus or a lentivirus.

In some embodiments, the RNA binding protein (RBP) comprises one or more of BTG1, CNOT2, CNOT4, CNOT7, CPSF5, DDX6, EWSR1, FUBP1, hnRNPAO, hnRNPC1/2, MEX3C, NANOS1, NANOS2, NOP56, PARN, PRR3, RBM14, RBM7, RPS6, SAMD4A, SNRPA, SRSF11, TOB1, TOB2, UTP11L, YTHDF2, ZC3H18, ZCCHC11, ZFP36, ZFP36L1, ZFP36L2, ABT1, AC004381.6, AIMP1, ALDH18A1, ANXA2, APOBEC3F, ASCC1, ATP5C1, BCCIP, BOLL, BYSL, BZW1, CELF5, CLK1, CLK2, CPSF1, DAZ2, DAZ3, DAZ4, DCN, DDX1, DDX19B, DDX20, DDX39A, DMPK, EEF1A1, EIF3G, ERAL1, XOSC4, FAM46A, FAM98A, FKBP3, FXR2, G3BP2, GLTSCR2, GSPT2, GTF2F1, GTPBP10, HADHB, HDGF, hnRNPEl, HNRPDL, HSPB1, KIAA1324, LARP1, LARP4, LARP4B, LIN28, LIN28A, LUC7L, MAK16, MATR3, MBNL2, MEPCE, MRPL39, MTDH, NDUFV3, NUFIP2, NUSAPI, PABPC1, PABPC5, PCBP4, PEG10, PPAN, PPIL4, PRPF3, PRPF31, PRRC2B, PTRH1, PUS7, RBM33, RBM38, RBMX2, RPL10A, RPL14, RPL15, RPLP0, RPS20, RPUSD3, RPUSD4, RTN4, SERBP1, SF3A3, SFRS10, SFRS13A, SFRS2IP, SLC7A9, SMN1, SPATS2L, SRSF5, SRSF8, THOC1, TRA2A, TRIM39, TUFM, UBAP2L, UTP23, XPO5, XRN1, YWHAE, and ZRANB2. In some embodiments, the RNA binding protein (RBP) is LIN28.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1F shows an RNA binding protein sponge construct competing for LIN28 binding. FIG. 1A shows a schematic diagram of the RNA binding agent (e.g., “Sponge”), wherein a DNA expression vector can be used to express the RNA binding agent, which is a single strand of RNA that contains a binding site for an RNA binding protein (RBP) of interest (central portion of transcript). This transcript is also protected on the 5′ and 3′ ends by an SCNMV Exo Element and a MALAT1 pseudoknot, respectively, which prevents its degradation by cellular nucleases. FIG. 1B shows electrophoretic mobility shift assay showing the association of LIN28A with biotin labeled let-7a with a Kd˜25 nM. FIG. 1C shows filter-binding assay showing the association of LIN28A with biotin labeled let-7a with a Kd˜IOnM. FIG. 1D shows electrophoretic mobility shift assay showing LIN28A in complex with biotin labeled let-7a and its dissociation by unlabeled let-7a competitor and sponge construct competitor. FIG. 1E shows electrophoretic mobility shift assay showing LIN28A in complex with biotin labeled let-7a and its dissociation by an unrelated shRNA construct designed to target YTHDF1, but not by polyadenylic acid (polyA RNA). FIG. 1F shows filter-binding assay showing the dissociation of biotin labeled let-7a from LIN28A in the presence of various competitors (let-7a, Sponge, Sponge Backbone, shRNA and polyA RNA).

FIGS. 2A-2F show expression of a LIN28 sponge construct alters gene expression in mouse embryonic stem cells. FIG. 2A shows binding sites that are shared before and after sponge expression. The heatmap compares the sites detected in condition M (vertical axis) to the sites in condition N (horizontal axis). FIG. 2B shows distribution of ratios that reflect changes in read density at all detected binding sites in response to the expression of a LIN28 sponge construct. FIG. 2C shows cumulative distribution functions showing the minimum free energy of the ensemble for all binding sites detected before sponge expression (N=992 sites) and after sponge expression (N=70 sites) compared to randomly chosen sequences outside of eCLIP-identified binding sites (Dashed Line). FIG. 2D shows a design of gene expression constructs used to track changes in let-7 miRNA activity in response to the depletion of LIN28 by sponge in single cells, as measured by analytical flow cytometry. Differences in miRNA activity are measured via changes in Turquoise-to-Cherry fluorescence as a function of sponge expression (Citrine). All fluorescence data is binned, and trends map the average response to LIN28B expression (N=3). FIG. 2E shows changes in polysome enrichment in V6.5 mESCs and Dicer knockout mESCs after the expression of LIN28 sponge. FIG. 2F shows a rolling mean plot showing the relationship between the change in polysome enrichment (rank ordered from the most repressed to the most activated by sponge construct) and the frequency of let-7 target sites per gene.

FIGS. 3A-3B shows the RBP binding agent (e.g., “Sponge”) competing for LIN28 binding and influencing miRNA regulation. FIG. 3A shows the ability of various potential competitor RNAs was examined using a bait pulldown assay. As visualized by Western blot, increasing the concentration of an unlabeled let-7a or sponge competitor displaced LIN28 from beads, whereas shRNA and polyA RNA could not.

FIG. 3B shows expression of the sponge competitor in V6.5 mESCs specifically influenced the expression of let-7 family miRNAs, while leaving the biogenesis of other miRNA families unaffected.

### DETAILED DESCRIPTION

This disclosure describes methods for controlling activity of a RNA binding protein (RBP) within a cell. Detailed herein are methods of inhibiting activity of a RNA binding protein in a cell that include (a) generating a RNA binding protein (RBP) binding agent comprising an RNA molecule comprising a binding site for the RNA binding protein; and (b) delivering the RBP binding agent into the cell, wherein the RBP binding agent binds to the RNA binding protein, thereby inhibiting the activity of the RNA binding protein in the cell Various non-limiting aspects of these methods are described herein, and can be used in any combination without limitation. Additional aspects of various components of methods for modulating gene expression are known in the art.

It must be noted that, as used in the specification and the appended claims, the singular forms “a,” “an” and “the” include plural referents unless the context clearly dictates otherwise.

As used herein, “affinity” refers to the strength of the sum total of non-covalent interactions between a ligand and its binding partner. Unless indicated otherwise, as used herein, “affinity” refers to intrinsic binding affinity, which reflects a 1:1 interaction between members of a ligand and a binding site. The affinity of a molecule X for its partner Y can be represented by the dissociation equilibrium constant (K_{D}). Affinity can be measured by common methods known in the art. Affinity can be determined, for example, using surface plasmon resonance (SPR) technology (e.g., BIACORE®) or biolayer interferometry (e.g., FORTEBIO®). Additional methods for determining the affinity for a ligand and its corresponding binding site are known in the art.

As used herein, “biological sample” can refer to a sample generally including cells and/or other biological material. A biological sample can be obtained from non-mammalian organisms (e.g., a plants, an insect, an arachnid, a nematode), a fungi, an amphibian, or a fish (e.g., zebrafish). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). Biological samples can be derived from a homogeneous culture or population of organisms or alternatively from a collection of several different organisms, for example, in a community or ecosystem.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins.

In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

As used herein, the term “plurality” can refer to a state of having a plural (e.g., more than one) number of different types of things (e.g., a cell, a genomic sequence, a subject, a system, or a protein). In some embodiments, a plurality of nucleic acid sequences can be more than one nucleic acid sequence wherein each nucleic acid sequence is different from each other. In other embodiments, “plurality” can refer to a state of having a plural number of the same thing (e.g., a cell, a genomic sequence, a subject, a system, or a protein). In some embodiments, a plurality of nucleic acid sequences are identical to each other. In some embodiments, a plurality of cells are cellular clones (e.g., identical cells).

### RNA Binding Protein (RBP)

RNA binding proteins are proteins that bind to the double or single stranded RNA in cells and have important roles in cellular processes (e.g., cellular transport or localization). RNA binding proteins (RBPs) play a major role in post-transcriptional control of RNAs (e.g., splicing, polyadenylation, mRNA stabilization, mRNA localization and translation). In some embodiments, an RBP is a cytoplasmic protein. In some embodiments, an RBP is a protein that interacts with RNA molecules (e.g., mRNA) from synthesis to decay to affect their metabolism, localization, stability, and translation. In some embodiments, an RBP is a nuclear protein. In some embodiments, RBPs can include, but are not limited to, splicing factors, RNA stability factors, histone stem-loop binding proteins, or ribosomes. For example, a eukaryotic ribosome can include a collection of RBPs that can interact directly with mRNA coding sequences.

In some embodiments, an RNA binding protein comprises a ribosomal protein, wherein the ribosomal protein binds to a ribosome and/or an mRNA during translation. In some embodiments, the RNA binding protein comprises at least one of: SLTM, ZGPAT, PPARGClB, PELP1, DCP2, CSTF3, TRA2B, ZNF638, SRSF9, LUC7L2, PTBP3, SF3B3, VCP, HNRNPA2B1, PTBP1, PCBP2, LSM14A, LSM12, DHX15, DDX27, DDX17, DDX21, IPO5, RPL22L1, RPL35, RPSA, MRPS34, NIFK, THUMPD1, RPUSD3, RRBP1, EEFSEC, UBAP2L, PUS7L, EIF4ENIF1, BICC1, EIF4E2, DARS2, TRDMT1, UPF3B, ZFP36L2, YTHDF2, EDC3, HNRNPR, UPF3A, ELAVL1, RBM27, XRN1, FUS, EXOSC7, PSPC1, CNOT7, CNOT6, CNOT4, CNOT3, AGO2, ENDOU, RBFOX1 (A2BP1), RBFOX2 (RBM9), RBFOX3 (NeuN), SLBP, RBM5, RBM6, PRBP1, ACO1, Adat1, PCBP1, PCBP3, PCBP4, RBM3, RBM4, APOBEC1, BTG1, CNOT2, CPSF5, DDX6, EWSR1, FUBP1, hnRNPAO, hnRNPC1/2, MEX3C, NANOS1, NANOS2, NOP56, PARN, PRR3, RBM14, RBM7, RPS6, SAMD4A, SNRPA, SRSF11, TOB1, TOB2, UTP11L, ZC3H18, ZCCHC11, ZFP36, ZFP36L1, ABT1, AC004381.6, AIMP1, ALDH18A1, ANXA2, APOBEC3F, ASCC1, ATP5C1, BCCIP, BOLL, BYSL, BZW1, CELF5, CLK1, CLK2, CPSF1, DAZ2, DAZ3, DAZ4, DCN, DDX1, DDX19B, DDX20, DDX39A, DMPK, EEF1A1, EIF3G, ERAL1, XOSC4, FAM46A, FAM98A, FKBP3, FXR2, G3BP2, GLTSCR2, GSPT2, GTF2F1, GTPBP10, HADHB, HDGF, hnRNPEl, HNRPDL, HSPB1, KIAA1324, LARP1, LARP4, LARP4B, LIN28A, LUC7L, MAK16, MATR3, MBNL2, MEPCE, MRPL39, MTDH, NDUFV3, NUFIP2, NUSAPI, PABPC1, PABPC5, PCBP4, PEG10, PPAN, PPIL4, PRPF3, PRPF31, PRRC2B, PTRH1, PUS7, RBM33, RBM38, RBMX2, RPL10A, RPL14, RPL15, RPLP0, RPS20, RPUSD3, RPUSD4, RTN4, SERBP1, SF3A3, SFRS10, SFRS13A, SFRS2IP, SLC7A9, SMN1, SPATS2L, SRSF5, SRSF8, THOC1, TRA2A, TRIM39, TUFM, UBAP2L, UTP23, XPO5, XRN1, YWHAE, or ZRANB2. In some embodiments, the RNA binding protein comprises A1CF, AAMP, AAR2, AARS, AARS2, AARSD1, AATF, ABCE1, ABCF1, ABCF3, ABT1, AC004381.6, ACAA2, ACIN1, ACO1, ACTN4, ADAD1, ADAD2, ADAR, ADARB1, ADARB2, ADAT1, ADAT2, ADAT3, ADD1, ADK, AEN, AFF1, AFF2, AFF3, AFF4, AGFG1, AGFG2, AGGF1, AGO1, AGO2, AGO3, AGO4, AHNAK, AICDA, AIMP1, AIMP2, AKAP1, AKAP13, AKAP17A, AKAP8, AKAP8L, ALDH18A1, ALDH6A1, ALKBH1, ALKBH5, ALKBH8, ALYREF, ANG, ANGEL1, ANGEL2, ANKHD1, ANKRD17, ANXA2, APEH, APEX1, APEX2, API5, APOBEC1, APOBEC2, APOBEC3A, APOBEC3B, APOBEC3C, APOBEC3D, APOBEC3F, APOBEC3G, APOBEC3H, APOBEC4, APTX, AQR, ARCN1, ARHGEF1, ARHGEF18, ARHGEF2, ARHGEF28, ARL6IP4, ARPP21, ASCC1, ASCC3, ASH1L, ASS1, ATG16L1, ATG16L2, ATP5A1, ATP5C1, ATXN1, ATXN1L, ATXN2, ATXN2L, AUH, AZGP1, BARD1, BAZ2A, BAZ2B, BCCIP, BCDIN3D, BCLAF1, BICC1, BLM, BMS1, BOLL, BOP1, BRCA1, BRIX1, BST2, BTF3, BTRC, BUB3, BUD13, BYSL, BZW1, BZW2, Cllorf68, C12orf65, C14orfl66, C14orf93, C15orf52, C16orf88, C17orf85, CID, C1QBP, Clorfl3l, Clorf35, Clorf52, C22orf28, C2orfl5, C4BPA, C6orf52, C7orf50, C9orfl 14, C9orfl29, CACTIN, CALR, CALR3, CANX, CAPRIN1, CAPRIN2, CARHSP1, CARS, CARS2, CASC3, CAST, CCAR1, CCAR2, CCDCl08, CCDCl24, CCDCl37, CCDC47, CCDC59, CCDC75, CCDC86, CCDC9, CCNC, CCNK, CCNL1, CCNL2, CCNT1, CCNT2, CCRN4L, CCT4, CCT6A, CD180, CD2BP2, CD3EAP, CDC40, CDC42EP4, CDC5L, CDK10, CDK11A, CDK11B, CDK12, CDK13, CDK5RAP1, CDK9, CDKAL1, CEBPZ, CELF1, CELF2, CELF3, CELF4, CELF5, CELF6, CENPI, CHD2, CHD3, CHERP, CHTOP, CIRBP, CIRHIA, CISD2, CKAP4, CLASRP, CLGN, CLK1, CLK2, CLK3, CLK4, CLNS1A, CLP1, CMSS1, CMTR1, CMTR2, CNBP, CNOT1, CNOT10, CNOT11, CNOT2, CNOT3, CNOT4, CNOT6, CNOT6L, CNOT7, CNOT8, CNP, COA6, COL14A1, CORO1A, CPEB1, CPEB2, CPEB3, CPEB4, CPNE3, CPSF1, CPSF2, CPSF3, CPSF3L, CPSF4, CPSF4L, CPSF6, CPSF7, CRKL, CRNKL1, CRYZ, CSDA, CSDC2, CSDE1, CSRP1, CSTB, CSTF1, CSTF2, CSTF2T, CSTF3, CTIF, CTNNA1, CTU1, CTU2, CWC15, CWC22, CWC25, CWC27, CWF19L1, CWF19L2, CXorf23, Cas9, DALRD3, DAP3, DARS, DARS2, DAW1, DAZ1, DAZ2, DAZ3, DAZ4, DAZAP1, DAZL, DBR1, DCAF13, DCD, DCN, DCP1A, DCP1B, DCP2, DCPS, DDB1, DDX1, DDX10, DDX17, DDX18, DDX19A, DDX19B, DDX20, DDX21, DDX23, DDX24, DDX25, DDX26B, DDX27, DDX28, DDX31, DDX39A, DDX39B, DDX3X, DDX3Y, DDX4, DDX41, DDX42, DDX43, DDX46, DDX47, DDX49, DDX5, DDX50, DDX51, DDX52, DDX53, DDX54, DDX55, DDX56, DDX58, DDX59, DDX6, DDX60, DDX60L, DEK, DENR, DGCR14, DGCR8, DHX15, DHX16, DHX29, DHX30, DHX32, DHX33, DHX34, DHX35, DHX36, DHX37, DHX38, DHX40, DHX57, DHX58, DHX8, DHX9, DIAPHI, DICERI, DIEXF, DIMT1, DIS3, DIS3L, DIS3L2, DKC1, DMGDH, DNAAF2, DNAH1, DNAH10, DNAH11, DNAH17, DNAH2, DNAH3, DNAH5, DNAH6, DNAH7, DNAH8, DNAH9, DNAJC12, DNAJC17, DNAJC2, DNAJC21, DNAL4, DND1, DNMT1, DNMT3A, DNMT3B, DNMT3L, DNTTIP2, DPPA5, DQX1, DRG1, DRG2, DROSHA, DSP, DUS1L, DUS2, DUS2L, DUS3L, DUS4L, DUSP11, DUT, DXO, DYNCIHI, DYNCILIl, DYNC2H1, DYNLL1, DYNLL2, DZIP1, DZIP1L, DZIP3, EARS2, EBNA1BP2, ECH1, ECHDC2, ECHDC3, ECHS1, EDC3, EDC4, EDF1, EED, EEF1A1, EEF1A2, EEF1B2, EEF1D, EEF1E1, EEF1G, EEF2, EEF2K, EEFSEC, EFTUD1, EFTUD2, EIF1, EIFIAD, EIFIAX, EIF1AY, EIF1B, EIF2A, EIF2AK1, EIF2AK2, EIF2AK3, EIF2AK4, EIF2B1, EIF2B2, EIF2B3, EIF2B4, EIF2B5, EIF2C1, EIF2C2, EIF2C3, EIF2C4, EIF2D, EIF2S1, EIF2S2, EIF2S3, EIF2S3L, EIF3A, EIF3B, EIF3C, EIF3CL, EIF3D, EIF3E, EIF3G, EIF3H, EIF3I, EIF3J, EIF3K, EIF3L, EIF3M, EIF4A1, EIF4A2, EIF4A3, EIF4B, EIF4E, EIF4ElB, EIF4E2, EIF4E3, EIF4ENIF1, EIF4G1, EIF4G2, EIF4G3, EIF4H, EIF5, EIF5A, EIF5A2, EIF5AL1, EIF5B, EIF6, ELAC1, ELAC2, ELAVL1, ELAVL2, ELAVL3, ELAVL4, EMG1, ENDOG, ENDOU, ENDOV, ENO1, ENOX1, ENOX2, EPRS, ERAL1, ERCC3, ERI1, ERI2, ERI3, ERN1, ERN2, ESF1, ESRP1, ESRP2, ETF1, EWSR1, EXO1, EXOG, EXOSC1, EXOSC10, EXOSC2, EXOSC3, EXOSC4, EXOSC5, EXOSC6, EXOSC7, EXOSC8, EXOSC9, EZH1, EZH2, EZR, FAM103A1, FAM120A, FAM120B, FAM120C, FAM208A, FAM32A, FAM46A, FAM46B, FAM46C, FAM46D, FAM58A, FAM98A, FAM98B, FAM98C, FANCM, FARS2, FARSA, FARSB, FASN, FASTK, FASTKD1, FASTKD2, FASTKD3, FASTKD5, FAU, FBL, FBLL1, FBXO17, FBXO2, FBXO27, FBXO44, FBXO6, FBXW11, FBXW2, FBXW7, FCF1, FCGRT, FDPS, FDXACB1, FIP1L1, FKBP3, FKBP4, FLNA, FLYWCH2, FMR1, FNDC3A, FNDC3B, FRG1, FRG1B, FSCN1, FTO, FTSJ1, FTSJ2, FTSJ3, FTSJD2, FUBP1, FUBP3, FUS, FXR1, FXR2, FYTTD1, G3BP1, G3BP2, GANAB, GAPDH, GAPDHS, GAR1, GARS, GATC, GCFC2, GEMIN2, GEMIN4, GEMIN5, GEMIN6, GEMIN7, GEMIN8, GFM1, GFM2, GLE1, GLRX3, GLTSCR2, GNB2L1, GNL1, GNL2, GNL3, GNL3L, GOLGB1, GPANK1, GPATCH1, GPATCH2, GPATCH3, GPATCH4, GPATCH8, GPKOW, GRB2, GRN, GRSF1, GRWD1, GSPT1, GSPT2, GTF2E2, GTF2F1, GTF3A, GTPBP1, GTPBP10, GTPBP2, GTPBP3, GTPBP4, GTSF1, GTSF1L, GUF1, H1F0, HABP4, HADHB, HARS, HARS2, HBP1, HBS1L, HDGF, HDGFL1, HDGFRP2, HDGFRP3, HDLBP, HEATR1, HELQ, HELZ, HELZ2, HENMT1, HERC5, HEXIM1, HEXIM2, HFE, HFM1, HIF1AN, HINT3, HIST1H1B, HIST1H1C, HISTTH4H, HLA-A, HLA-B, HLA-C, HLA-E, HLA-F, HLA-G, HLTF, HMGB1, HMGB2, HNRNPA0, HNRNPA1, HNRNPA1L2, HNRNPA2B1, HNRNPA3, HNRNPAB, HNRNPC, HNRNPCL1, HNRNPD, HNRNPDL, HNRNPF, HNRNPH1, HNRNPH2, HNRNPH3, HNRNPK, HNRNPL, HNRNPLL, HNRNPM, HNRNPR, HNRNPU, HNRNPUL1, HNRNPUL2, HNRPDL, HNRPLL, HRSP12, HSP90AA1, HSP90AB1, HSPA1B, HSPA8, HSPA9, HSPB1, HSPBAP1, HSPD1, HSPE1, HTATSF1, HUWEl, IARS, IARS2, ICT1, IFI16, IFIH1, IFIT1, IFIT1B, IFIT2, IFIT3, IFIT5, IGF2BP1, IGF2BP2, IGF2BP3, IGHMBP2, ILF2, ILF3, IMMT, IMP3, IMP4, IMPDHT, INTS1, INTS10, INTS12, INTS2, INTS3, INTS4, INTS5, INTS6, INTS7, INTS8, INTS9, IPO11, IP013, IP04, IPO5, IP07, IP08, IP09, IREB2, IRP1, IRP2, ISG20, ISG20L2, ISYl, JAKMIP1, JAKMIP2, JAKMIP3, KARS, KAT5, KAT6A, KAT6B, KAT7, KAT8, KHDC1, KHDC1L, KHDRBS1, KHDRBS2, KHDRBS3, KHNYN, KHSRP, KIAA0020, KIAA0391, KIAA0430, KIAA1324, KIAA1456, KIAA1967, KIF1C, KIN, KPNB1, KRCC1, KRI1, KRR1, KRT18, KTN1, L1TD1, LARP1, LARPIB, LARP4, LARP4B, LARP6, LARP7, LARS, LARS2, LAS1L, LCMT1, LCMT2, LENG9, LGALS1, LGALS3, LIN28A, LIN28B, LLPH, LONP1, LONP2, LRP1, LRPPRC, LRRC47, LRRC59, LRRFIP1, LRRFIP2, LSG1, LSM1, LSM10, LSM11, LSM12, LSM14A, LSM14B, LSM2, LSM3, LSM4, LSM5, LSM6, LSM7, LSMD1, LTA4H, LUC7L, LUC7L2, LUC7L3, LUZP4, LYAR, MAEL, MAGOH, MAGOH3P, MAGOHB, MAK16, MAP4, MARK2, MARS, MARS2, MATR3, MAZ, MBNL1, MBNL2, MBNL3, MCTS1, MDH2, MECP2, MEPCE, METAP2, METTL1, METTL10, METTL14, METTL16, METTL25, METTL2A, METTL2B, METTL3, METTL5, METTL6, METTL8, MEX3A, MEX3B, MEX3C, MEX3D, MFAP1, MIF4GD, MKI67, MKI67IP, MKRN1, MKRN2, MKRN3, MLL3, MOV10, MOV10L1, MPHOSPH10, MPHOSPH6, MPST, MR1, MRM1, MRP63, MRPL1, MRPL10, MRPL11, MRPL12, MRPL13, MRPL14, MRPL15, MRPL16, MRPL17, MRPL18, MRPL19, MRPL2, MRPL20, MRPL21, MRPL22, MRPL23, MRPL24, MRPL27, MRPL28, MRPL3, MRPL30, MRPL32, MRPL33, MRPL34, MRPL35, MRPL36, MRPL37, MRPL38, MRPL39, MRPL4, MRPL40, MRPL41, MRPL42, MRPL43, MRPL44, MRPL45, MRPL46, MRPL47, MRPL48, MRPL49, MRPL50, MRPL51, MRPL52, MRPL53, MRPL54, MRPL55, MRPL9, MRPS10, MRPS11, MRPS12, MRPS14, MRPS15, MRPS16, MRPS17, MRPS18A, MRPS18B, MRPS18C, MRPS2, MRPS21, MRPS22, MRPS23, MRPS24, MRPS25, MRPS26, MRPS27, MRPS28, MRPS30, MRPS31, MRPS33, MRPS34, MRPS35, MRPS36, MRPS5, MRPS6, MRPS7, MRPS9, MRRF, MRTO4, MSI1, MSI2, MSL3, MTDH, MTERFD2, MTFMT, MTG1, MTG2, MTHFSD, MTIF2, MTIF3, MTO1, MTPAP, MTRF1, MTRF1L, MURC, MVP, MYBBPlA, MYEF2, N4BP1, NA, NAA15, NAA38, NAF1, NANOS1, NANOS2, NANOS3, NAP1L4, NARS, NARS2, NAT10, NCBP1, NCBP2, NCBP2L, NCCRP1, NCL, NDUFV3, NELFE, NFX1, NFXL1, NGDN, NGRN, NHP2, NHP2L1, NIFK, NIP7, NIPBL, NKRF, NLRP11, NMD3, NME1, NOA1, NOB1, NOC2L, NOC3L, NOC4L, NOL10, NOL11, NOL12, NOL3, NOL6, NOL7, NOL8, NOL9, NOLC1, NOM1, NONO, NOP10, NOP14, NOP16, NOP2, NOP56, NOP58, NOP9, NOSIP, NOVA1, NOVA2, NPM1, NPM2, NPM3, NQO1, NROB1, NROB2, NSA2, NSRP1, NSUN2, NSUN3, NSUN4, NSUN5, NSUN6, NSUN7, NUDT16, NUDT16L1, NUDT21, NUFIP1, NUFIP2, NUP153, NUP35, NUPL2, NUSAPI, NUTF2, NVL, NXF1, NXF2, NXF2B, NXF3, NXF5, NXT1, NXT2, NYNRIN, OAS1, OAS2, OAS3, OASL, OBFC1, P4HB, PA2G4, PABPC1, PABPC1L, PABPC1L2A, PABPC1L2B, PABPC3, PABPC4, PABPC4L, PABPC5, PABPN1, PABPN1L, PAIP1, PAIP2, PAIP2B, PAN2, PAN3, PAPD4, PAPD5, PAPD7, PAPOLA, PAPOLB, PAPOLG, PARK7, PARN, PARP1, PARP10, PARP11, PARP12, PARP14, PARP15, PARP2, PARP3, PARP4, PARS2, PATL1, PATL2, PATZ1, PAXBP1, PCBP1, PCBP2, PCBP3, PCBP4, PCF11, PCSK9, PDCD11, PDCD4, PDCD7, PDE12, PDIA3, PDIA4, PEBP1, PEBP4, PEG10, PELO, PES1, PET112, PHAX, PHF5A, PHF6, PHRF1, PIH1D1, PIH1D2, PIH1D3, PIN4, PINX1, PIWIL1, PIWIL2, PIWIL3, PIWIL4, PKM, PKN2, PLD6, PLEC, PLRG1, PNLDC1, PNN, PNO1, PNPT1, PNRC1, PNRC2, POLDIP3, POLK, POLQ, POLR1B, POLR1E, POLR2A, POLR2B, POLR2D, POLR2E, POLR2F, POLR2G, POLR2H, POLR2I, POLR2J, POLR2J2, POLR2J3, POLR2K, POLR2L, POLR3A, POLR3B, POLR3E, POLRMT, POP1, POP4, POP5, POP7, POU5F1, PPAN, PPARGC1A, PPARGClB, PPHLN1, PPIA, PPIAL4A, PPIAL4B, PPIAL4C, PPIAL4D, PPIAL4G, PPIB, PPIC, PPIE, PPIF, PPIG, PPIH, PPIL1, PPIL2, PPIL3, PPIL4, PPP1R10, PPP1R8, PPRC1, PPWD1, PQBP1, PRDX1, PRDX2, PRDX3, PRDX4, PRIM1, PRKCDBP, PRKDC, PRKRA, PRPF18, PRPF19, PRPF3, PRPF31, PRPF38A, PRPF38B, PRPF39, PRPF4, PRPF40A, PRPF40B, PRPF4B, PRPF6, PRPF8, PRR3, PRRC2B, PRRC2C, PSIP1, PSMA1, PSMA6, PSMC1, PSMD4, PSPC1, PSTK, PTBP1, PTBP2, PTBP3, PTCD1, PTCD2, PTCD3, PTGES3, PTGES3L, PTGES3L-AARSD1, PTPN1, PTRF, PTRH1, PTRH2, PTRHD1, PUF60, PUM1, PUM2, PURA, PURB, PURG, PUS1, PUS10, PUS3, PUS7, PUS7L, PUSL1, PWP2, QARS, QKI, QRSL1, QTRT1, QTRTD1, R3HCC1, R3HCC1L, R3HDM1, R3HDM2, RAE1, RALY, RALYL, RAN, RANBP17, RANBP2, RANBP6, RARS, RARS2, RAVER1, RAVER2, RBBP6, RBFOX1, RBFOX2, RBFOX3, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM3, RBM33, RBM34, RBM38, RBM39, RBM4, RBM41, RBM42, RBM43, RBM44, RBM45, RBM46, RBM47, RBM48, RBM4B, RBM5, RBM6, RBM7, RBM8A, RBMS1, RBMS2, RBMS3, RBMX, RBMX2, RBMXL1, RBMXL2, RBMXL3, RBMYlA1, RBMYlB, RBMYlD, RBMY1E, RBMYlF, RBMYlJ, RBPMS, RBPMS2, RC3H1, RC3H2, RCC2, RCL1, RDBP, RDM1, RDX, RECQL, RECQL4, RECQL5, REPINI, REXO1, REXO2, REXO4, RGPD1, RGPD2, RGPD3, RGPD4, RGPD5, RGPD6, RGPD8, RIMS1, RIOK1, RIOK2, RIOK3, RNASE1, RNASE10, RNASE11, RNASE12, RNASE13, RNASE2, RNASE3, RNASE4, RNASE6, RNASE7, RNASE8, RNASE9, RNASEH1, RNASEH2A, RNASEH2B, RNASEH2C, RNASEK, RNASEL, RNASET2, RNF113A, RNF113B, RNF17, RNGTT, RNH1, RNMT, RNMTL1, RNPC3, RNPS1, RP9, RPF1, RPF2, RPGR, RPL10, RPL10A, RPL10L, RPL11, RPL12, RPL13, RPL13A, RPL14, RPL15, RPL17, RPL18, RPL18A, RPL19, RPL21, RPL22, RPL22L1, RPL23, RPL23A, RPL24, RPL26, RPL26L1, RPL27, RPL27A, RPL28, RPL29, RPL3, RPL30, RPL31, RPL32, RPL34, RPL35, RPL35A, RPL36, RPL36A, RPL36AL, RPL37, RPL37A, RPL38, RPL39, RPL39L, RPL3L, RPL4, RPL41, RPL5, RPL6, RPL7, RPL7A, RPL7AP10, RPL7L1, RPL8, RPL9, RPLP0, RPLP1, RPLP2, RPN1, RPP14, RPP21, RPP25, RPP25L, RPP30, RPP38, RPP40, RPS10, RPS11, RPS12, RPS13, RPS14, RPS15, RPS15A, RPS16, RPS17, RPS17L, RPS18, RPS19, RPS19BP1, RPS2, RPS20, RPS21, RPS23, RPS24, RPS25, RPS26, RPS26P32, RPS27, RPS27A, RPS27L, RPS28, RPS29, RPS3, RPS3A, RPS4X, RPS4Y1, RPS4Y2, RPS5, RPS6, RPS7, RPS8, RPS9, RPSA, RPSAP58, RPUSD1, RPUSD2, RPUSD3, RPUSD4, RQCD1, RRBP1, RRNAD1, RRP1, RRP12, RRP15, RRP1B, RRP36, RRP7A, RRP8, RRP9, RRS1, RSL1D1, RSL24D1, RSRC1, RTCA, RTCB, RTF1, RTN4, RUVBL1, RUVBL2, S100A16, S100A4, SAFB, SAFB2, SAMD4A, SAMD4B, SAMHD1, SAMSN1, SAP18, SARNP, SARS, SARS2, SART1, SART3, SBDS, SCAFI, SCAF11, SCAF4, SCAF8, SCG3, SDAD1, SDPR, SEC23IP, SEC61B, SEC63, SECISBP2, SECISBP2L, SEPSECS, SERBP1, SERPINHI, SETBP1, SETD1A, SETD1B, SETD7, SETX, SF1, SF3A1, SF3A2, SF3A3, SF3B1, SF3B14, SF3B2, SF3B3, SF3B4, SF3B5, SFPQ, SFSWAP, SHQ1, SIDT1, SIDT2, SKIV2L, SKIV2L2, SLBP, SLC16A3, SLC25A5, SLC35G6, SLC3A2, SLC4A1AP, SLIRP, SLTM, SLU7, SMAD1, SMAD2, SMAD3, SMAD4, SMAD5, SMAD6, SMAD7, SMAD9, SMG1, SMG5, SMG6, SMG7, SMG8, SMG9, SMN1, SMN2, SMNDC1, SND1, SNIP1, SNORD84, SNRNP200, SNRNP25, SNRNP27, SNRNP35, SNRNP40, SNRNP48, SNRNP70, SNRPA, SNRPA1, SNRPB, SNRPB2, SNRPC, SNRPD1, SNRPD2, SNRPD3, SNRPE, SNRPEP2, SNRPF, SNRPG, SNRPN, SNTB2, SNUPN, SNW1, SOGA2, SON, SORBS2, SPAG16, SPATS2, SPATS2L, SPEN, SRA1, SRBD1, SREK1, SRFBP1, SRP14, SRP19, SRP54, SRP68, SRP72, SRP9, SRPK1, SRPK2, SRPK3, SRPR, SRRM1, SRRM2, SRRM3, SRRM4, SRRT, SRSF1, SRSF10, SRSF11, SRSF12, SRSF2, SRSF3, SRSF4, SRSF5, SRSF6, SRSF7, SRSF8, SRSF9, SSB, SSBP1, SSRP1, SSU72, STAU1, STAU2, STIP1, STK31, STRAP, STRBP, STXBP1, SUB1, SUCLG1, SUGP1, SUGP2, SUMO1, SUPT16H, SUPT4H1, SUPT5H, SUPT6H, SUPV3L1, SURF6, SUV39H1, SUV39H2, SUZ12, SWT1, SYF2, SYMPK, SYNCRIP, SYNE1, TAF15, TAF9, TAF9B, TARBP1, TARBP2, TARDBP, TARS, TARS2, TARSL2, TBL2, TBL3, TBRG4, TCERG1, TCERG1L, TCF20, TCOF1, TDRD1, TDRD10, TDRD12, TDRD15, TDRD3, TDRD5, TDRD6, TDRD7, TDRD9, TDRKH, TEFM, TEP1, TERT, TES, TEX13A, TEX13B, TFAM, TFB1M, TFB2M, TFIP11, TFRC, TGS1, THG1L, THOC1, THOC2, THOC3, THOC5, THOC6, THOC7, THRAP3, THUMPD1, THUMPD2, THUMPD3, TIAl, TIAL1, TIPARP, TLR3, TLR4, TLR5, TLR7, TLR8, TLR9, TMSB4X, TNPO1, TNPO2, TNPO3, TNRC6A, TNRC6B, TNRC6C, TNS1, TOE1, TOP1, TOP1MT, TOP3A, TOP3B, TOPAZI, TP5313, TPD52L2, TPR, TPT1, TRA2A, TRA2B, TRAP1, TRDMT1, TRIM11, TRIM16, TRIM16L, TRIM17, TRIM2, TRIM21, TRIM22, TRIM25, TRIM27, TRIM3, TRIM34, TRIM38, TRIM39, TRIM4, TRIM45, TRIM47, TRIM5, TRIM56, TRIM58, TRIM6, TRIM60, TRIM65, TRIM68, TRIM7, TRIM71, TRIMLI, TRIP6, TRIT1, TRMT1, TRMT10A, TRMT10B, TRMT10C, TRMT11, TRMT112, TRMT12, TRMT13, TRMT1L, TRMT2A, TRMT2B, TRMT44, TRMT5, TRMT6, TRMT61A, TRMT61B, TRMU, TRNAU1AP, TRNT1, TROVE2, TRPT1, TRUB1, TRUB2, TSEN15, TSEN2, TSEN34, TSEN54, TSFM, TSN, TSNAX, TSR1, TSR2, TSR3, TST, TTF2, TUFM, TUT1, TWF2, TXN, TXNL4A, TXNL4B, TYW1, TYW1B, TYW3, TYW5, U2AF1, U2AF1L4, U2AF2, U2SURP, UBA1, UBA52, UBA6, UBA7, UBAP2, UBAP2L, UBB, UBBP4, UBC, UBE2I, UBE2L3, UBFD1, UBTF, UCHL5, UHMK1, UNK, UNKL, UPF1, UPF2, UPF3A, UPF3B, URB1, URB2, URM1, USB1, USO1, USP10, USP36, USP39, UTP11L, UTP14A, UTP14C, UTP15, UTP18, UTP20, UTP23, UTP3, UTP6, VARS, VARS2, VARSL, VAT1, VAT1L, VEZF1, VWA5A, WARS, WARS2, WBP4, WBSCR16, WDR12, WDR25, WDR3, WDR36, WDR37, WDR38, WDR4, WDR43, WDR46, WDR5, WDR5B, WDR6, WDR61, WDR75, WDR83, WDR86, WHSC1, WHSC1L1, WIBG, WRAP53, WRN, XAB2, XIRP1, XPO1, XPO4, XPO5, XPO6, XPO7, XPOT, XRCC5, XRCC6, XRN1, XRN2, YARS, YARS2, YBX1, YBX2, YBX3, YRDC, YTHDC1, YTHDC2, YTHDF1, YTHDF2, YTHDF3, YWHAE, YWHAG, YWHAZ, ZADH2, ZC3H10, ZC3H11A, ZC3H12A, ZC3H12B, ZC3H12C, ZC3H12D, ZC3H13, ZC3H14, ZC3H15, ZC3H18, ZC3H3, ZC3H4, ZC3H6, ZC3H7A, ZC3H7B, ZC3H8, ZC3HAV1, ZC3HAV1L, ZC3HC1, ZCCHC11, ZCCHC13, ZCCHC14, ZCCHC16, ZCCHC17, ZCCHC2, ZCCHC24, ZCCHC3, ZCCHC5, ZCCHC6, ZCCHC7, ZCCHC8, ZCCHC9, ZCRB1, ZFAND4, ZFC3H1, ZFP106, ZFP36, ZFP36L1, ZFP36L2, ZFP62, ZFR, ZFR2, ZGPAT, ZMAT1, ZMAT2, ZMAT3, ZMAT4, ZMAT5, ZNF106, ZNF107, ZNF160, ZNF197, ZNF239, ZNF268, ZNF326, ZNF346, ZNF347, ZNF385A, ZNF385B, ZNF385C, ZNF385D, ZNF43, ZNF473, ZNF493, ZNF546, ZNF579, ZNF585A, ZNF585B, ZNF594, ZNF598, ZNF616, ZNF622, ZNF624, ZNF629, ZNF638, ZNF658, ZNF664, ZNF721, ZNF768, ZNF780B, ZNF800, ZNF804A, ZNF804B, ZNF808, ZNF814, ZNF836, ZNF841, ZNF845, ZNF99, ZNFX1, ZNHIT6, ZRANB2, ZRSR1, ZRSR2, ZYX. In some embodiments, the RNA binding protein comprises BTG1, CNOT2, CNOT4, CNOT7, CPSF5, DDX6, EWSR1, FUBP1, hnRNPAO, hnRNPC1/2, MEX3C, NANOS1, NANOS2, NOP56, PARN, PRR3, RBM14, RBM7, RPS6, SAMD4A, SNRPA, SRSF11, TOB1, TOB2, UTP11L, YTHDF2, ZC3H18, ZCCHC11, ZFP36, ZFP36L1, ZFP36L2, ABT1, AC004381.6, AIMP1, ALDH18A1, ANXA2, APOBEC3F, ASCC1, ATP5C1, BCCIP, BOLL, BYSL, BZW1, CELF5, CLK1, CLK2, CPSF1, DAZ2, DAZ3, DAZ4, DCN, DDX1, DDX19B, DDX20, DDX39A, DMPK, EEF1A1, EIF3G, ERAL1, XOSC4, FAM46A, FAM98A, FKBP3, FXR2, G3BP2, GLTSCR2, GSPT2, GTF2F1, GTPBP10, HADHB, HDGF, hnRNPEl, HNRPDL, HSPB1, KIAA1324, LARP1, LARP4, LARP4B, LIN28, LIN28A, LUC7L, MAK16, MATR3, MBNL2, MEPCE, MRPL39, MTDH, NDUFV3, NUFIP2, NUSAPI, PABPC1, PABPC5, PCBP4, PEG10, PPAN, PPIL4, PRPF3, PRPF31, PRRC2B, PTRH1, PUS7, RBM33, RBM38, RBMX2, RPL10A, RPL14, RPL15, RPLP0, RPS20, RPUSD3, RPUSD4, RTN4, SERBP1, SF3A3, SFRS10, SFRS13A, SFRS2IP, SLC7A9, SMN1, SPATS2L, SRSF5, SRSF8, THOC1, TRA2A, TRIM39, TUFM, UBAP2L, UTP23, XPO5, XRN1, YWHAE, or ZRANB2. In some embodiments, the RNA binding protein is LIN28.

RNA-binding proteins (RBPs) have roles in controlling the fate of RNAs including the modulation of pre-mRNA splicing, RNA modification, translation, stability, and localization. RBPs are a group of proteins that interact with RNA using an array of strategies from well-defined RNA-binding domains to disordered regions that recognize RNA sequence and/or secondary structures.

### RBP Binding Agent

Provided herein are methods of inhibiting activity of a RNA binding protein (RBP) in a cell, the method including: (a) generating a RBP binding agent comprising an RNA molecule comprising a binding site for the RNA binding protein; and (b) delivering the RBP binding agent into the cell, wherein the RBP binding agent binds to the RNA binding protein, thereby inhibiting the activity of the RNA binding protein in the cell.

As used herein, “RBP binding agent” can refer to an agent that specifically binds to a RNA binding protein (RBP) of interest. In some embodiments, the RBP binding agent comprises an RNA molecule. In some embodiments, the RNA molecule is a single strand RNA. In some embodiments, the RBP binding agent can also be referred to as an RBP binding “sponge”.

In some embodiments, the RBP binding agent comprises a binding site for the RNA binding protein. In some embodiments, the RBP binding agent comprises one or more binding sites for the RNA binding protein. In some embodiments, the RNA binding protein binds to the one or more binding sites with low affinity. In some embodiments, the RNA binding protein binds to the one or more binding sites with high affinity. Low affinity binding can refer to a relatively high concentration of a ligand being required before the corresponding binding site is maximally occupied. High affinity binding can refer to a relatively low concentration of a ligand being adequate to maximally occupy a ligand-binding site.

In some embodiments, the RBP binding agent comprises a single binding site for a single RNA binding protein. In some embodiments, the RBP binding agent comprises a plurality of binding sites. In some embodiments, the plurality of binding sites can bind one or more of a same RNA binding protein. In some embodiments, the plurality of binding sites can bind one or more of different RNA binding proteins. In some embodiments, the RBP binding agent comprises a plurality of binding sites for a plurality of the same RNA binding proteins. In some embodiments, the RBP binding agent comprises a plurality of binding sites for a plurality of different RNA binding proteins.

In some embodiments, the RBP binding agent further comprises a pseudoknot at each of the 5′ and 3′ ends. As used herein, a “pseudoknot” can refer to a nucleic acid secondary structure containing at least two stem-loop structures in which half of one stem is intercalated between the two halves of another stem. A pseudoknot is an RNA structure that is minimally composed of two helical segments connected by singe-stranded regions or loops. Due to variation of the lengths of the loops and stems, as well as the types of interactions between them, pseudoknots represent a structurally diverse group having a variety of diverse roles that include forming the catalytic core of various ribozymes, self-splicing introns, and telomerase. In some embodiments, pseudoknots can alter gene expression by inducing ribosomal frameshifting. In some embodiments, pseudoknots can confer nuclease resistance. In some embodiments, pseudoknots can promote accumulations of RBP binding agents in a cell. In some embodiments, the RBP binding agent further comprises a SCNMV Exo element and a MALAT1 Pseudoknot, respectively on the 5′ and 3′ ends.

Method of Inhibiting Activity of an RNA Binding Protein Provided herein are methods of inhibiting activity of a RNA binding protein in a cell that include (a) generating a RNA binding protein (RBP) binding agent comprising an RNA molecule comprising a binding site for the RNA binding protein; and (b) delivering the RBP binding agent into the cell, wherein the RBP binding agent binds to the RNA binding protein, thereby inhibiting the activity of the RNA binding protein in the cell.

In some embodiments, the RBP binding agent can be delivered into a host cell by a variety of well-known techniques. In some embodiments, the delivery method can include a vector-mediated gene transfer (e.g., viral infection/transfection, or various other protein-based or lipid-based gene delivery complexes). In some embodiments, the delivery method can include methods facilitating the delivery of “naked” polynucleotides (e.g., electroporation, “gene gun” delivery and various other techniques used for the introduction of polynucleotides). In some embodiments, the delivering comprises transfection, electroporation, or a virus-based delivery. In some embodiments, the delivering comprises a virus-based delivery. In some embodiments, the virus-based delivery comprises an adeno-associated virus or a lentivirus.

### Vectors

In some embodiments of the methods disclosed herein, a vector comprises a RBP binding agent of the present disclosure. In some embodiments, the RBP binding agent comprises a synthetic gene. As used herein, a “synthetic gene” can refer to a foreign nucleic sequence that is artificially engineered and/or assembled. In some embodiments, a synthetic gene can be an exogenous gene. In some embodiments, the RBP binding agent is generated by using a DNA expression vector. In some embodiments, the DNA expression vector comprises a promoter. In some embodiments, the promoter is a TRE promoter.

In some embodiments, a vector is a viral vector. In some embodiments, the viral vector includes a sequence isolated or derived from a retrovirus. In some embodiments, the viral vector includes a sequence isolated or derived from a lentivirus. In some embodiments, the viral vector includes a sequence isolated or derived from an adenovirus. In some embodiments, the viral vector includes a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments, the viral vector includes a sequence isolated or derived from an adeno-associated virus (AAV). In some embodiments, the viral vector includes an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, AAV12, AAV.rh32/33, AAV.rh43, AAV.rh64R1, and any combinations or equivalents thereof. In some embodiments, the viral vector is replication incompetent. In some embodiments, the viral vector is isolated or recombinant (rAAV). In some embodiments, the viral vector is self-complementary (scAAV). In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb.

In some embodiments, a vector is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. In some embodiments, the promoter is a TRE promoter.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVsM) vector, a modified sooty mangabey simian immunodeficiency virus (SIVsM) vector, a African green monkey simian immunodeficiency virus (SIVAGm) vector, a modified African green monkey simian immunodeficiency virus (SIVAGm) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### EXAMPLES

The disclosure is further described in the following examples, which do not limit the scope of the disclosure described in the claims.

### Example 1—Non-Mirna Binding Sites Sequester Lin28 Protein

LIN28 does not need to directly bind a transcript to impart post-transcriptional regulation. Non-miRNA binding sites can still influence gene expression indirectly by sequestering LIN28 protein from miRNAs. The vast number of non-miRNA sites on coding and ribosomal transcripts could act as binding decoys and could prevent LIN28 from regulating pre/pri-miRNA biogenesis. For this to be true, non-miRNAs must be bound by LIN28 as strongly as miRNAs, which would allow them to be competitive inhibitors. Sequestration of LIN28 by this mechanism could buffer global changes in miRNA activity.

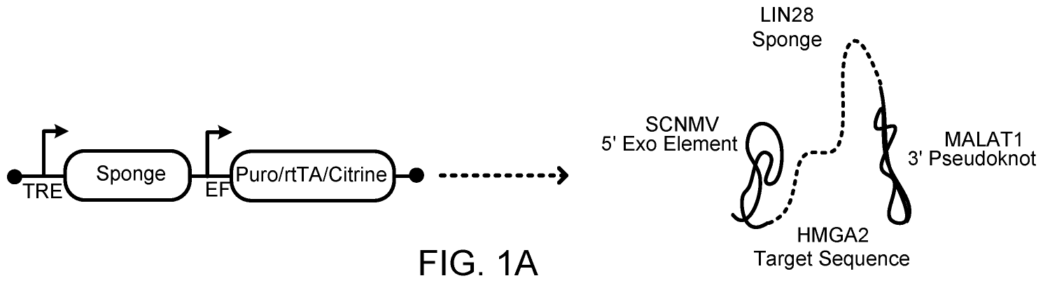

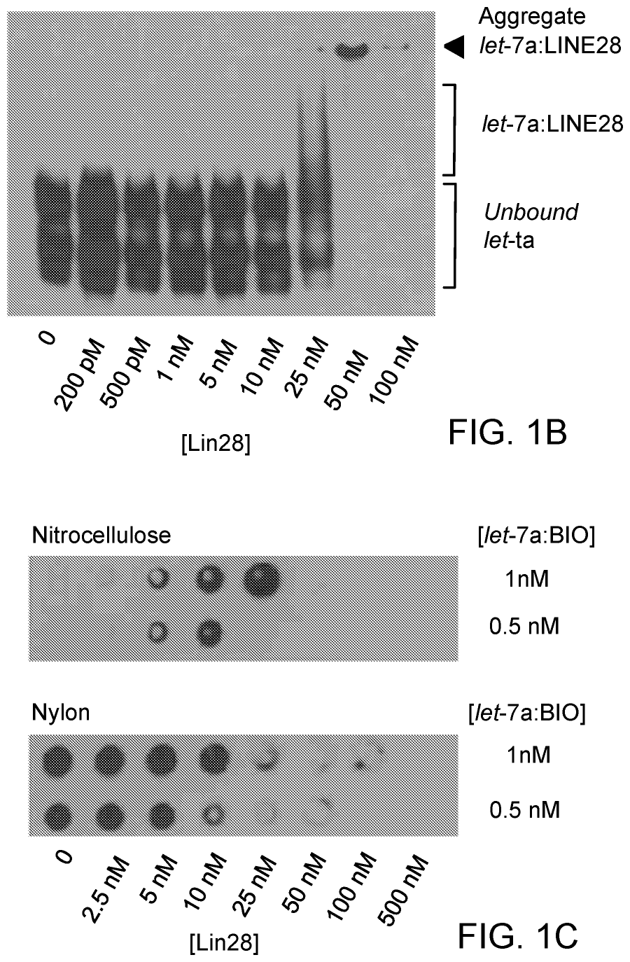

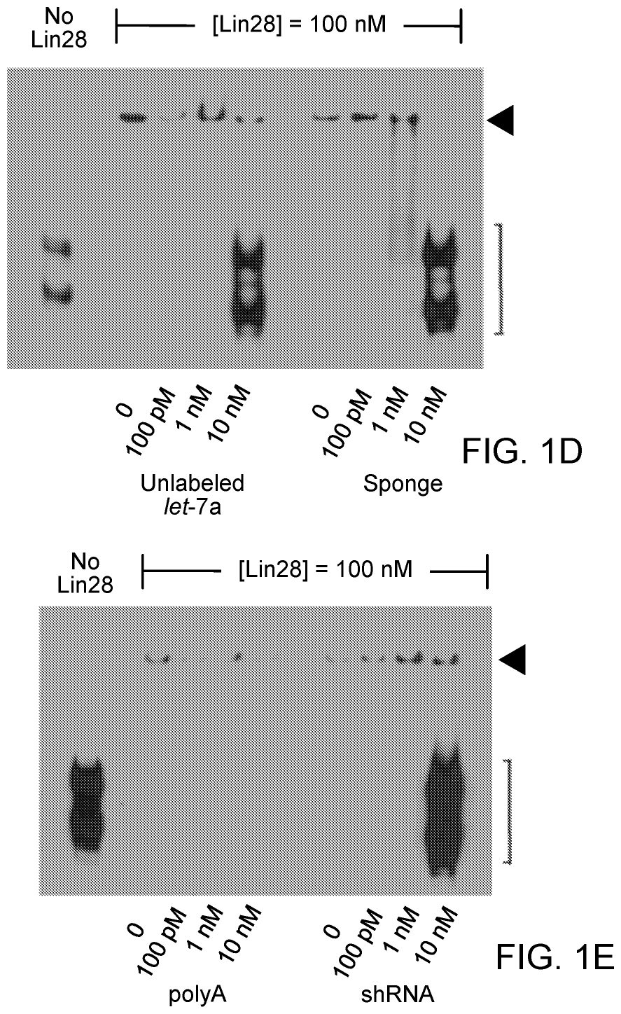

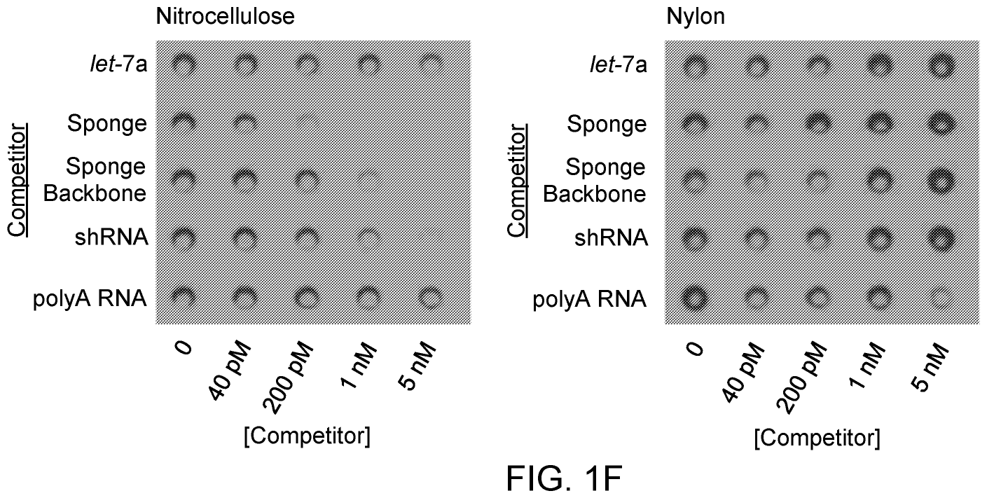

To test this hypothesis, the balance of miRNA and non-miRNA binding in a cell was disrupted and the effect of this disruption on gene regulation was examined. A non-coding RNA that could be expressed from an inducible transgene to bind and sequester LIN28 protein was constructed (FIG. 1A). This molecular sponge construct was modeled after a high affinity target site detected in the Human HMGA2 3′UTR, which binds LIN28 preferentially at low concentrations. Pseudoknots were also incorporated at the 5′ and 3′ ends to confer nuclease resistance and to promote the accumulation of sponge in cells.

It was confirmed that LIN28A can bind the sponge construct. Experiments showed that LIN28A binds pre-let-7a miRNA with nanomolar affinity (Kd ˜25 nM) using an electrophoretic mobility shift assay (EMSA) (FIG. 1B). Given the propensity of LIN28A to form higher order ribonucleoprotein complexes above 50 nM, which tend to aggregate in the well of a polyacrylamide gel, this association was also characterized using a filter binding assay (FIG. 1C). A filter binding assay employs a sandwich of three membranes through which a sample is passed under vacuum, with each membrane screening for a specific type of protein-RNA interaction. The first layer polyethersulfone; PES) retains all higher order aggregated ribonucleoprotein complexes. The second layer (nitrocellulose) retains free protein and any RNA bound to these proteins. The third layer (nylon) retains all free RNA. Using this system, a transition between unbound and bound pre-let-7a miRNA was seen when LIN28A reaches a concentration ˜10 nM. Like the EMSA, LIN28A concentrations 50 nM and above caused pre-let-7a miRNA to be retained in the PES layer as an aggregate, as shown by the lack of any signal on nitrocellulose or nylon.

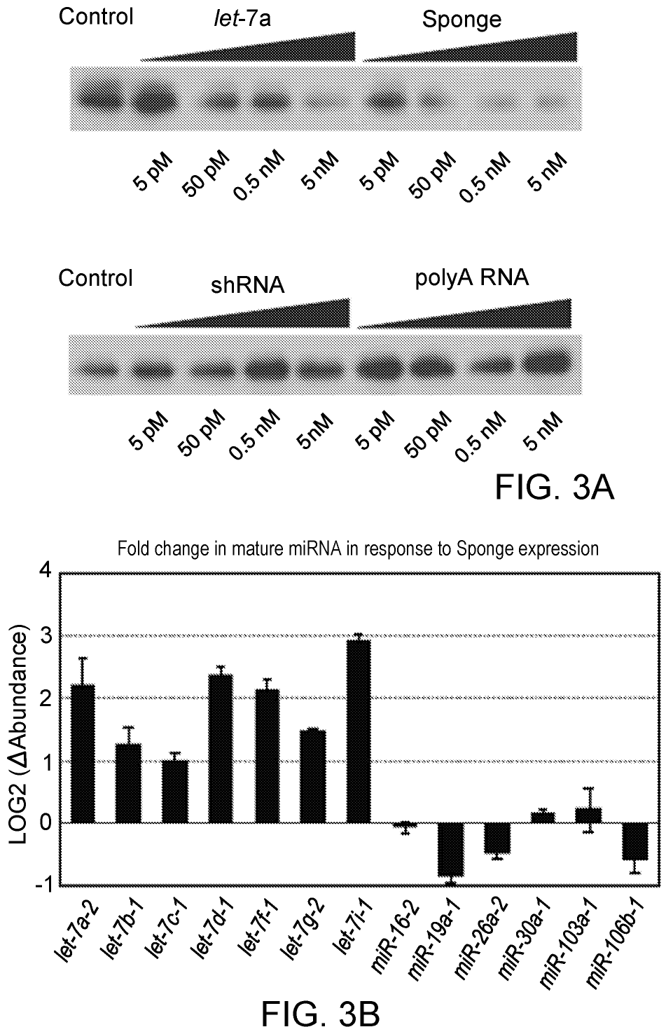

The LIN28 sponge construct serves as an effective competitor for LIN28A binding, as shown by the displacement of pre-let-7a miRNA from LIN28A when present at roughly equivalent concentrations (lnM and above) (FIG. 1D). Interestingly, a random short hairpin RNA construct used as a control was also found to displace pre-let-7a in a gel shift assay (FIG. 1E), even though the same construct could not influence LIN28 binding when pre-let-7a was presented as a tethered bait (FIG. 3A). This short hairpin construct contains “GNNG” sequences and is also distinguished by a well-defined hairpin structure, which could explain its ability to interact with LIN28A, albeit inconsistently. By contrast, repetitive and unstructured polyadenylic acid (polyA) with an average molecular weight of 250 kDa could not displace LIN28A from pre-let-7 miRNA (FIG. 1D).

A filter binding assay largely reproduces these gel shift results, showing the depletion of probe from nitrocellulose and its accumulation on nylon in the presence of competitors (unlabeled let-7a, sponge, shRNA), but not in the presence of polyA RNA (FIG. 1F). Interestingly, these data indicate the sponge may bind LIN28A more strongly than pre-let-7a miRNA. The generic binding motifs and the preference of LIN28 for structured RNA also permits the protective elements at the 5′ and 3′ ends to play a role in siphoning LIN28 protein. The sponge backbone (absent any LIN28 binding site) can compete as effectively as unlabeled pre-let-7a miRNA (FIG. 1F). Nonetheless, the sponge appears to be specific for LIN28 as evidenced by significant changes in mature let-7 expression (FIG. 3B).

### Example 2—Sequestration of Lin28 Protein Alters Mirna Regulation

It was next examined whether the sponge construct, when expressed in cells, could be used to disrupt LIN28 activity. The sponge was stably expressed in mouse embryonic stem cells (V6.5 mESCs), a cell type that maintains high levels of Lin28A, and it was confirmed that sponge expression enhanced the biogenesis of let-7 family miRNAs (FIG. 3B). eCLIP sequencing libraries were created to monitor changes in transcriptome-wide Lin28A occupancy and polysome sequencing libraries were created to track changes in gene expression in response to sponge expression.

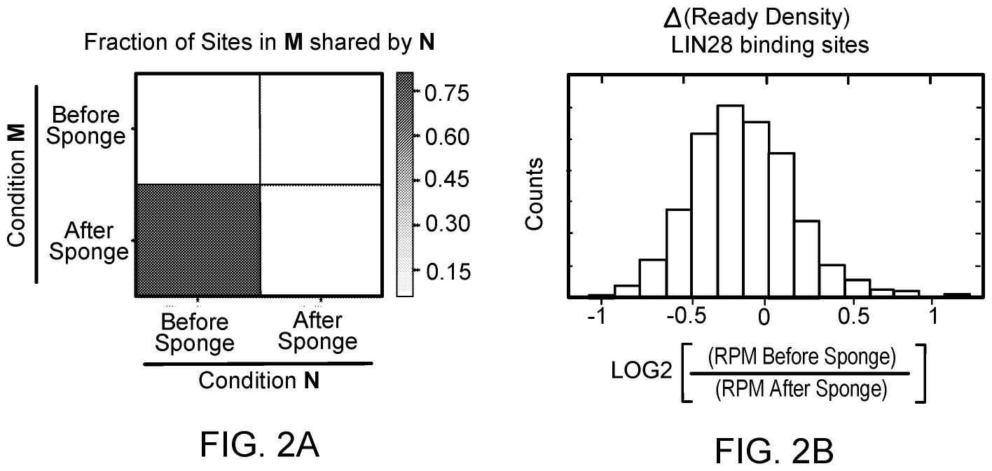

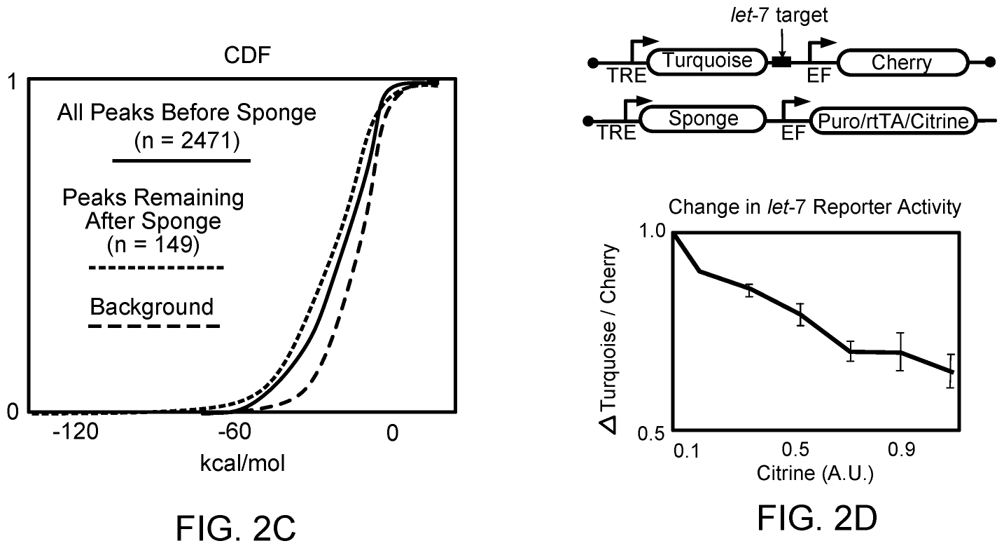

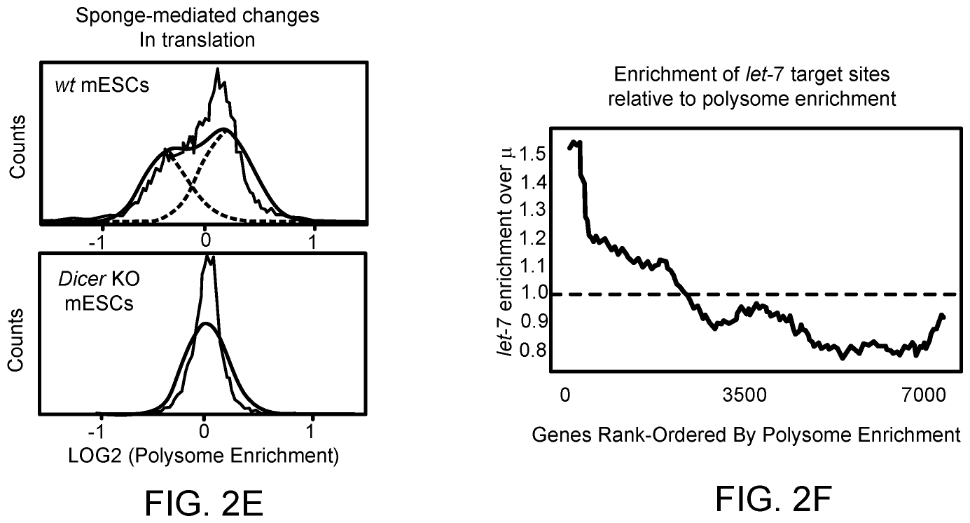

1449 binding sites were detected before sponge expression and 185 binding sites after sponge expression, with an overlap of 131 sites (71%) (FIG. 2A). The reduction in detectable binding sites after sponge expression was expected, since sequestration of LIN28 by the sponge would preferentially impact occupancy at lower affinity sites, resulting in relatively more frequent binding at higher affinity sites, and a redistribution of eCLIP sequencing reads (FIG. 2B). Indeed, it was confirmed that binding sites detected after sponge expression had a greater potential to form structure (i.e., lower free energy) than sites detected in the absence of sponge (FIG. 2C).

Importantly, by displacing LIN28 from pri-/pre-let-7 miRNAs, the sponge competitor caused an increase in cellular let-7 miRNA activity, as gauged using a fluorescent reporter with a fully complementary let-7 miRNA target site (FIG. 2D). These changes in miRNA activity permitted the separation of the wt mESCs transcriptome into two translational subpopulations (FIG. 2E). It was shown that these LIN28-dependent subpopulations are associated with genes that are either enriched for let-7 target sites (repressed genes) or genes which are enriched for all other non-let-7 miRNA target sites (activated genes). By contrast, in the absence of miRNA activity (i.e., Dicer knockout), it was found that sponge expression had no effect on gene expression (FIG. 2E). These data are consistent with the role of non-miRNA binding sites as competitors for LIN28 binding, which primarily affect gene expression by modulating miRNA pathway activity.

Sequestration of LIN28 protein indirectly regulates gene expression by enhancing let-7 miRNA biogenesis. The changes in gene expression observed in wt mESCs are consistent with an increase in let-7 miRNA activity, as transcripts enriched in let-7 family target sites are repressed (high rank) (FIG. 2F). And given rate limiting expression of miRNA pathway components, increased let-7 activity negatively impacts the ability of all other non-let-7 miRNAs to regulate their target genes. Consequently, the expression of transcripts enriched in non-let-7 miRNA target sites is enhanced (low rank) (FIG. 2F). Together, these observations underscore the central importance of miRNAs in mediating LIN28 regulation and the ability of non-miRNA binding to modulate their activity.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inhibiting activity of a RNA binding protein (RBP) in a cell, the method comprising:
(a) generating a RNA binding protein (RBP) binding agent comprising an RNA molecule comprising a binding site for the RNA binding protein; and
(b) delivering the RBP binding agent into the cell, wherein the RBP binding agent binds to the RNA binding protein, thereby inhibiting the activity of the RNA binding protein in the cell.

2. The method of claim 1, wherein the RNA molecule is a single strand RNA.

3. The method of claim 1, wherein the RBP binding agent comprises one or more binding sites for the RNA binding protein.

4. The method of claim 3, wherein the RNA binding protein binds to the one or more binding sites with low affinity.

5. The method of claim 3, wherein the RNA binding protein binds to the one or more binding sites with high affinity.

6. The method of claim 1, wherein the RBP binding agent comprises a single binding site for a single RNA binding protein.

7. The method of claim 1, wherein the RBP binding agent comprises a plurality of binding sites for a plurality of the same RNA binding protein.

8. The method of claim 1, wherein the RBP binding agent comprises a plurality of binding sites for a plurality of different RNA binding proteins.

9. The method of claim 1, wherein the RBP binding agent further comprises a pseudoknot at each of the 5′ and 3′ ends.

10. The method of claim 9, wherein the RBP binding agent further comprises a SCNMV Exo element and a MALAT1 Pseudoknot, respectively on the 5′ and 3′ ends.

11. The method of claim 1, wherein the RBP binding agent comprises a synthetic gene.

12. The method of claim 1, wherein the RBP binding agent is expressed by using a DNA expression vector.

13. The method of claim 12, wherein the DNA expression vector comprises a promoter.

14. The method of claim 13, wherein the promoter is a TRE promoter.

15. The method of claim 1, wherein the delivering comprises transfection, electroporation, or a virus-based delivery.

16. The method of claim 1, wherein the delivering comprises a virus-based delivery.

17. The method of claim 16, wherein the virus-based delivery comprises an adeno-associated virus or a lentivirus.

18. The method of claim 1, wherein the RNA binding protein (RBP) comprises one or more of BTG1, CNOT2, CNOT4, CNOT7, CPSF5, DDX6, EWSR1, FUBP1, hnRNPAO, hnRNPC1/2, MEX3C, NANOS1, NANOS2, NOP56, PARN, PRR3, RBM14, RBM7, RPS6, SAMD4A, SNRPA, SRSF11, TOB1, TOB2, UTP11L, YTHDF2, ZC3H18, ZCCHC11, ZFP36, ZFP36L1, ZFP36L2, ABT1, AC004381.6, AIMP1, ALDH18A1, ANXA2, APOBEC3F, ASCC1, ATP5C1, BCCIP, BOLL, BYSL, BZW1, CELF5, CLK1, CLK2, CPSF1, DAZ2, DAZ3, DAZ4, DCN, DDX1, DDX19B, DDX20, DDX39A, DMPK, EEF1A1, EIF3G, ERAL1, XOSC4, FAM46A, FAM98A, FKBP3, FXR2, G3BP2, GLTSCR2, GSPT2, GTF2F1, GTPBP10, HADHB, HDGF, hnRNPE1, HNRPDL, HSPB1, KIAA1324, LARP1, LARP4, LARP4B, LIN28, LIN28A, LUC7L, MAK16, MATR3, MBNL2, MEPCE, MRPL39, MTDH, NDUFV3, NUFIP2, NUSAP1, PABPC1, PABPC5, PCBP4, PEG10, PPAN, PPIL4, PRPF3, PRPF31, PRRC2B, PTRH1, PUS7, RBM33, RBM38, RBMX2, RPL10A, RPL14, RPL15, RPLP0, RPS20, RPUSD3, RPUSD4, RTN4, SERBP1, SF3A3, SFRS10, SFRS13A, SFRS2IP, SLC7A9, SMN1, SPATS2L, SRSF5, SRSF8, THOC1, TRA2A, TRIM39, TUFM, UBAP2L, UTP23, XPO5, XRN1, YWHAE, and ZRANB2.

19. The method of claim 18, wherein the RNA binding protein (RBP) is LIN28.
